(19) United States
(12) Patent Application Publication
(10) Pub. No.: US 2024/0181229 A1
(43) Pub. Date: Jun. 6, 2024

(54) BALLOON CATHETER AND BALLOON DILATATION METHOD

(71) Applicant: Japan Lifeline Co., Ltd., Tokyo (JP)

(72) Inventor: Kenji MORI, Tokyo (JP)

(21) Appl. No.: 18/483,659

(22) Filed: Oct. 10, 2023

(30) Foreign Application Priority Data
Oct. 13, 2022 (JP) 2022-164734
Aug. 21, 2023 (JP) 2023-134120

(51) Int. Cl. A61M 29/02; A61M 25/10

(52) U.S. Cl. CPC A61M 29/02; A61M 25/1002; A61M 2025/1084; A61M 2029/025

(57) ABSTRACT

A balloon catheter includes a shaft that extends in an axial direction from a proximal end side to a distal end side, a balloon that is attached to the distal end side of the shaft and is expandable by a fluid supplied from the proximal end side of the shaft, a distal end portion expansion lumen that is provided inside the shaft to supply the fluid to a distal end portion in the balloon, and a proximal end portion expansion lumen that is provided inside the shaft to supply the fluid to a proximal end portion in the balloon. The balloon expands, by the fluid simultaneously supplied from the distal end portion expansion lumen and the proximal end portion expansion lumen, into an hourglass shape having a narrow portion in an intermediate portion between the distal end portion and the proximal end portion.

### CROSS REFERENCE TO RELATED APPLICATIONS

This application claims the benefit of priority to Japanese Patent Application Numbers 2022-164734 and 2023-134120 filed on Oct. 13, 2022 and Aug. 21, 2023, respectively. The entire contents of each of the above-identified applications are hereby incorporated by reference.

### TECHNICAL FIELD

The present disclosure relates to a balloon catheter and the like.

### BACKGROUND

A catheter is a medical tube that is inserted into the body for inspection or treatment. In particular, a catheter including a balloon that is expandable in the body is referred to as a balloon catheter. A balloon catheter is used for dilating a dilatation target part or a constricted part in: a tubular organ in the body such as a blood vessel, a trachea, a digestive tract, a common bile duct, and a pancreatic duct, or a connection part (inlet and outlet) therebetween; a hole formed in the body for inspection or treatment (for example, a hole that is punctured in the common bile duct from the stomach or the duodenal bulb); and the like (for example, PLT 1: JP 2014-124264 A).

### SUMMARY

In a case of expanding a balloon disposed so as to span a dilatation target part, there is the concern that the balloon is subjected to a force that pushes back the balloon, separating the balloon from the dilatation target part, when the balloon starts to expand from a proximal end side and comes into contact with the dilatation target part, for example.

The present disclosure has been made in view of such circumstances, and an object thereof is to provide a balloon catheter and the like that can effectively dilate a dilatation target part.

In order to solve the above problem, a balloon catheter according to an aspect of the present disclosure includes: a shaft that extends in an axial direction from a proximal end side to a distal end side; and a balloon that is attached to the distal end side of the shaft and is expandable by a fluid supplied from the proximal end side of the shaft, wherein the shaft internally includes a distal end portion expansion lumen for supplying the fluid to a distal end portion in the balloon and a proximal end portion expansion lumen for supplying the fluid to a proximal end portion in the balloon.

Another aspect of the present disclosure relates to a balloon expansion method. In this method, fluid is simultaneously supplied from the distal end portion and the proximal end portion into the balloon to expand the balloon into an hourglass shape having a narrow portion in the intermediate portion between the distal end portion and the proximal end portion.

Note that optional combinations of the aforementioned components and those obtained by converting these expressions into methods, apparatuses, systems, recording media, computer programs, and the like are also included in the present disclosure.

According to a balloon catheter and the like of the present disclosure, a dilatation target part can be effectively dilated.

### BRIEF DESCRIPTION OF DRAWINGS

FIG. 1 schematically illustrates an overview of EPBD in which a papilla is a dilatation target.

FIG. 2 is a side view schematically illustrating an outer appearance of a balloon catheter.

FIG. 3 is a perspective view schematically illustrating a configuration of a distal end side of a shaft to which a balloon is attached.

FIG. 4 schematically illustrates a state in which the balloon is positioned with respect to the papilla.

FIG. 5 schematically illustrates a modification of an expansion lumen.

FIG. 6 schematically illustrates a modification of the shaft.

FIG. 7 schematically illustrates a state in which the balloon is positioned with respect to a constricted part.

FIG. 8 schematically illustrates an example of a balloon molding tool.

FIG. 9 is a cross-sectional view of a balloon catheter according to a second embodiment.

FIG. 10 is a cross-sectional view along line I-I in FIG. 9.

### DESCRIPTION OF EMBODIMENTS

Hereinafter, aspects for realizing the present disclosure (hereinafter also referred to as embodiments) will be described in detail with reference to the drawings. In the description and/or drawings, the same or equivalent constituent elements, members, and processing operations, and the like are denoted by the same reference numerals, and redundant descriptions are omitted. The scales and shapes of the illustrated parts are set for convenience to simplify the descriptions and should not be construed in a limiting manner unless otherwise specified. The embodiments are illustrative and do not limit the scope of the present disclosure in any way. Not all features or combinations of the features described in the embodiments are essential to the present disclosure.

In a first embodiment, a balloon catheter used in Endoscopic Papillary Balloon Dilatation (EPBD) for dilating a papilla (papilla of Vater or duodenal papilla) as a dilatation target part will be described as an example.

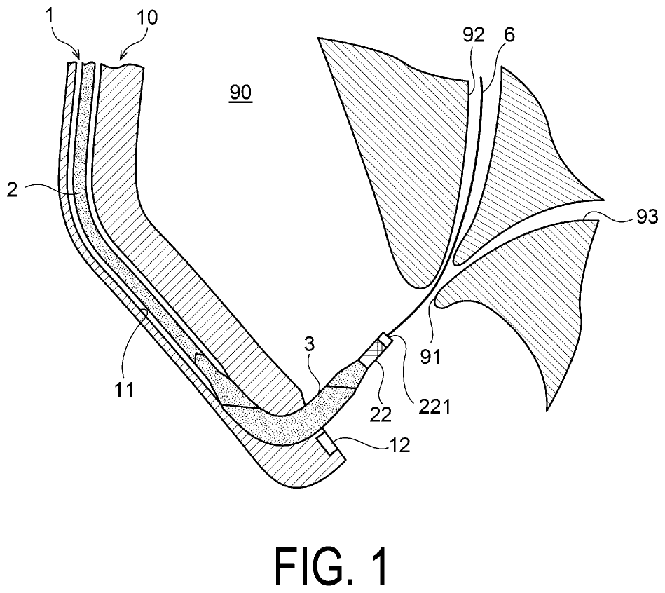

FIG. 1 schematically illustrates an overview of EPBD in which a papilla 91 is a dilatation target. An endoscope 10 includes a forceps channel 11 and a camera 12. The endoscope 10 is inserted into a duodenum 90 via the mouth. A balloon catheter 1 inserted into the body through the forceps channel 11 includes a tubular shaft 2 and a balloon 3 attached to a distal end side portion (a portion at the duodenum 90 side or a portion inside the body) of the tubular shaft 2. The balloon 3 is expandable by an expansion fluid obtained by appropriately mixing a contrast agent with sterile distilled water or saline supplied from a proximal end side of the balloon 3 (mouth side or extracorporeal side). Note that another liquid or gas such as air may be used as the expansion fluid if necessary for the purpose of inspection or treatment.

Prior to delivering the balloon catheter 1 to a target site (inspection site or treatment site), a guide wire 6 with a small diameter is inserted through the forceps channel 11 into a common bile duct 92 or a pancreatic duct 93 as a path leading to the target site. The papilla 91 is located, as a dilatation target part or an opening, between the duodenum 90, the common bile duct 92, and the pancreatic duct 93. The guide wire 6 with a sufficiently small diameter as compared to the opening diameter of the papilla 91 can pass through the papilla 91 and enter the inside of the common bile duct 92 or the pancreatic duct 93. At this time, an operator of the endoscope 10 can insert the guide wire 6 into the papilla 91 while checking an image obtained from the camera 12 disposed so as to face the papilla 91.

A long wire lumen (hole) penetrating from a proximal end portion to a distal end portion and through which the guide wire 6 can be inserted is formed inside the shaft 2. Prior to arranging the balloon catheter 1 in the papilla 91, the guide wire 6 is inserted into the common bile duct 92 or the pancreatic duct 93 from the duodenum 90 via the papilla 91. By inserting a distal end portion (an open end 221) of a wire lumen to be described later of the shaft 2 from a proximal end portion of the guide wire 6 at the extracorporeal side, the balloon 3 at the distal end portion of the shaft 2 guided by the guide wire 6 is directed to the papilla 91. In the state illustrated in the drawing, the shaft 2 is further moved forward along the guide wire 6, and the balloon 3 that has reached the position of the papilla 91 is expanded by the expansion fluid, dilating the papilla 91 from an inner side. By expanding the balloon 3 within the papilla 91, the papilla 91, which is normally constricted by the sphincter of Oddi (or the biliopancreatic ampulla sphincter), is dilated. Dilating the papilla 91 makes it possible to effectively remove a common bile duct calculus formed in the common bile duct 92 through the papilla 91, for example.

After the papilla 91 is dilated, the balloon 3 is contracted by discharging the expansion fluid to the outside of the body, and is taken out of the body together with the shaft 2 via the forceps channel 11 of the endoscope 10. After the balloon catheter 1 has been taken out from the body, a medical instrument such as another forceps or a cholangioscope for another medical procedure (e.g., moving a common bile duct stone from the papilla 91 into the duodenum 90 or to outside of the body) is inserted, if necessary, into the common bile duct 92 or the pancreatic duct 93 through the dilated papilla 91 while being guided by the guide wire 6.

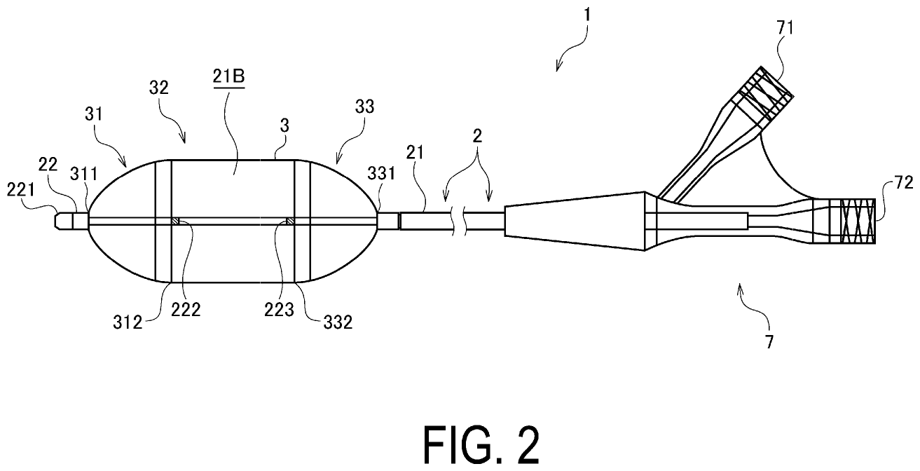

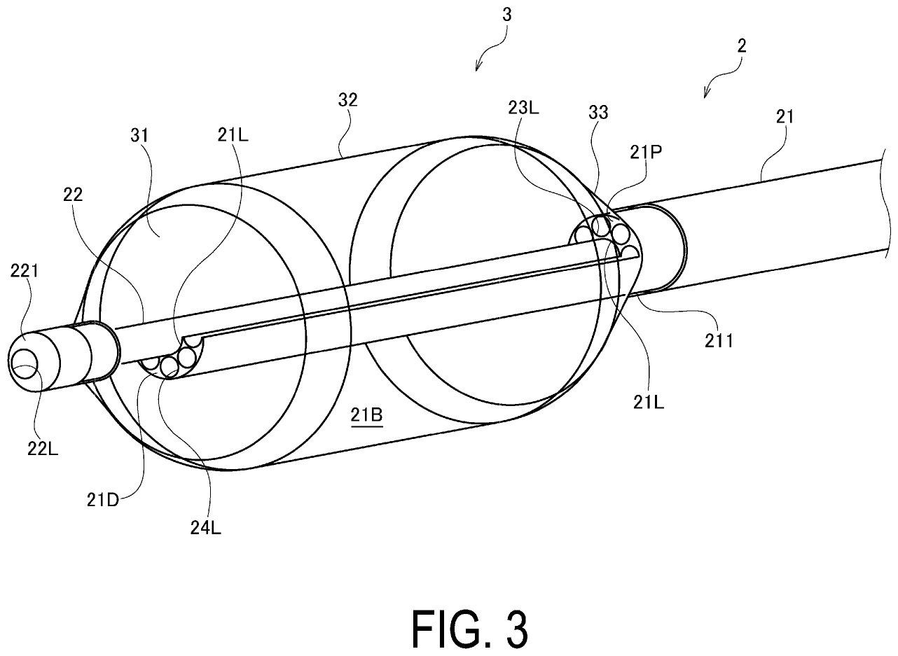

FIG. 2 is a side view schematically illustrating an outer appearance of the balloon catheter 1 according to the present embodiment. The balloon 3 is attached to a distal end portion (left end portion) of the flexible shaft 2 inside the body extending in an axial direction (lateral direction) from a proximal end side (right side in FIG. 2) toward a distal end side (left side in FIG. 2). FIG. 3 is a perspective view schematically illustrating a configuration of the distal end side of the shaft 2 to which the balloon 3 is attached. FIGS. 2 and 3 illustrate the balloon 3 in a fully expanded state thereof.

As illustrated in FIG. 2, the shaft 2 includes a tubular outer shaft 21 (an outer tube constituting the shaft 2) and a tubular inner shaft 22 (an inner tube constituting the shaft 2) in which a wire lumen through which the guide wire 6 passes is provided. In the balloon catheter 1, the balloon 3 includes three portions different from each other in an expanded state or expanded form, namely, a distal end side tapered portion 31, an intermediate portion 32, and a proximal end side tapered portion 33 in this order from the distal end toward the proximal end. The distal end side tapered portion 31 is formed in a tapered shape in which the maximum expansion diameter increases from a distal end portion 311 having substantially the same diameter as the outer periphery of the inner shaft 22 toward the intermediate portion 32 at the proximal end side. The proximal end side tapered portion 33 is formed in a tapered shape in which the maximum expansion diameter increases from a proximal end portion 331 having substantially the same diameter as the outer periphery of the outer shaft 21 toward the intermediate portion 32 on the distal end side.

In the illustrated example, in a direction connecting the distal end (the open end 221) and the proximal end (guide wire port 72) of the shaft 2 (the inner shaft 22) (hereinafter, also referred to as an axial direction, a longitudinal direction, or a left-right direction, and the dimension in this direction will be also referred to as a length), the length of the distal end side tapered portion 31 and the length of the proximal end side tapered portion 33 are substantially equal but may be significantly different from each other. In the illustrated example, in a direction of choice orthogonal to the axial direction (hereinafter, also referred to as a radial direction, an expansion direction, or an orthogonal direction, and the dimension in this direction will be also referred to as a diameter or an expansion diameter), the expansion diameter of the distal end side tapered portion 31 and the expansion diameter of the proximal end side tapered portion 33 respectively at points equally distant from the distal end portion 311 and the proximal end portion 331 in the axial direction are substantially equal (in other words, the inclination of the distal end side tapered portion 31 and the inclination of the proximal end side tapered portion 33 are substantially equal) but may be significantly different from each other. In the illustrated example where the maximum expansion diameter of a proximal end portion 312 of the distal end side tapered portion 31 and the maximum expansion diameter of a distal end portion 332 of the proximal end side tapered portion 33 are substantially the same, the intermediate portion 32 coupling these portions in the axial direction serves as a straight tube portion having a substantially uniform maximum expansion diameter.

A port portion 7 is provided at the proximal end side or the extracorporeal side of the shaft 2 (the outer shaft 21). The port portion 7 is provided with an expansion fluid port 71 and a guide wire port 72.

A fluid for expanding the balloon 3, specifically, an expansion fluid obtained by appropriately mixing a contrast agent with sterile distilled water or saline is supplied to the expansion fluid port 71. As illustrated in FIG. 3, the expansion fluid port 71 communicates with an internal space 21B of the balloon 3 via expansion lumens (a proximal end portion expansion lumen 23L and a distal end portion expansion lumen 24L) provided inside the outer shaft 21. When the expansion fluid is supplied from the expansion fluid port 71 to the internal space 21B, the balloon 3 is expanded. When the expansion fluid in the internal space 21B is discharged from the expansion fluid port 71, the balloon 3 contracted. The expansion fluid port 71 may be individually provided for the distal end portion expansion lumen 24L and the proximal end portion expansion lumen 23L among the expansion lumens provided inside the outer shaft 21. In this case, the distal end portion of the balloon 3 is preferentially expanded by the expansion fluid supplied from the distal end portion expansion fluid port communicating with the distal end portion expansion lumen 24L, and the proximal end portion of the balloon 3 is preferentially expanded by the expansion fluid supplied from the proximal end portion expansion fluid port communicating with the proximal end portion expansion lumen 23L.

The guide wire 6 is inserted into the guide wire port 72 to guide the balloon 3 to a target site, such as the papilla 91, as described above with respect to FIG. 1. The guide wire port 72 communicates with a wire lumen to be described later provided inside the inner shaft 22. The inner shaft 22 passes through a central lumen (described later) of the outer shaft 21 at the proximal end side of the shaft 2 where the balloon 3 is not provided. In the balloon 3, since the outer shaft 21 terminates stepwise as described later, the shaft 2 is only the inner shaft 22 in the end at the distal end portion thereof (distal end side tapered portion 31). The inner shaft 22 extends through the internal space 21B of the balloon 3 in the axial direction and terminates at the open end 221 which is the distal end of the entire balloon catheter 1. In this manner, an axial wire lumen through which the guide wire 6 can be inserted is formed inside the inner shaft 22 so as to penetrate from the guide wire port 72 at the proximal end portion to the open end 221 at the distal end portion. As described above with reference to FIG. 1, in a state where the guide wire 6 is inserted through the papilla 91 into the common bile duct 92 or the pancreatic duct 93, the open end 221 of the inner shaft 22 that is the distal end portion of the wire lumen is inserted from the proximal end portion of the guide wire 6 at the extracorporeal side so that the balloon 3 at the distal end portion of the shaft 2 guided by the guide wire 6 moves toward the papilla 91.

In order to facilitate positioning of the balloon 3 with respect to the papilla 91, a pair of contrast markers 222 and 223 provided on the outer periphery of the outer shaft 21 at axial positions corresponding to both end portions (312 and 332) of the intermediate portion 32 of the balloon 3 may be used. The position of the balloon 3 with respect to the papilla 91 can be checked in real time by checking the positions of the contrast markers 222 and 223 during the EPBD procedure.

A part of the shaft 2 extending from the balloon 3 to the proximal end side is constituted by the outer shaft 21 on the radially outer side and the inner shaft 22 on the radially inner side. To be more specific, the inner shaft 22 having a small tubular shape is passed through a central lumen 21L or a main lumen formed at the center in the radial direction inside the outer shaft 21 having a large tubular shape. As described above, the inner shaft 22 passes through the balloon 3 in the axial direction and extends to the open end 221. A wire lumen 22L through which the guide wire 6 passes is formed inside the inner shaft 22. The proximal end portion of the balloon 3 is attached to the outer periphery of a balloon attachment portion 211 at the distal end side of the outer shaft 21. It is possible to prevent the formation of a step between the proximal end portion of the balloon 3 attached to the balloon attachment portion 211 and the outer shaft 21 at the proximal end side by making the outer diameter of the balloon attachment portion 211 smaller than the outer diameter of the other portion of the outer shaft 21 by an amount corresponding to the thickness of the balloon 3.

In the axial direction, a part of the outer shaft 21 terminates at a proximal end surface 21P in the proximal end portion (the proximal end side of the intermediate portion 32 or the proximal end side tapered portion 33) of the balloon 3, and another part of the outer shaft 21 terminates at a distal end surface 21D in the distal end portion (the distal end side of the intermediate portion 32 or the distal end side tapered portion 31) of the balloon 3. In other words, the outer shaft 21 has two terminal end surfaces provided at different positions in the axial direction in the balloon 3. In the illustrated example, half of the outer shaft 21 having an annular shape (the central lumen 21L is formed in the center thereof and the inner shaft 22 passes through the central lumen 21L) as viewed in the axial direction terminates at the proximal end surface 21P, and the other half terminates at the distal end surface 21D. Thus, in an axial direction section between the proximal end surface 21P and the distal end surface 21D, only half of the outer shaft 21 extends as a cross section thereof as viewed in the axial direction extends. In this axial direction section, half of the outer periphery of the inner shaft 22 is supported by half of the outer shaft 21 having a semi-annular shape as viewed in the axial direction. Hereinafter, in the outer shaft 21 as viewed in the axial direction, a portion where the proximal end surface 21P is provided will be also referred to as an upper half or a northern hemisphere, and a portion where the distal end surface 21D is provided will be also referred to as a lower half or a southern hemisphere.

In the proximal end surface 21P (northern hemisphere), the distal end of one or more proximal end portion expansion lumens 23L opens into the proximal end portion of the balloon 3. Similarly, in the distal end surface 21D (southern hemisphere), the distal end of one or more distal end portion expansion lumens 24L opens into the distal end portion of the balloon 3. In the illustrated example, a plurality of proximal end portion expansion lumens 23L are formed along the circumferential direction so as to surround the central lumen 21L at the center in the northern hemisphere of the outer shaft 21. A plurality of distal end portion expansion lumens 24L are formed along the circumferential direction so as to surround the central lumen 21L at the center in the southern hemisphere of the outer shaft 21. Therefore, in the shaft 2 at the proximal end side of the balloon 3 constituted by the completely annular outer shaft 21 and the central inner shaft 22, the outer periphery of the central lumen 21L at the center is surrounded by the plurality of proximal end portion expansion lumens 23L and the plurality of distal end portion expansion lumens 24L.

The proximal end portions of the proximal end portion expansion lumen 23L and the distal end portion expansion lumen 24L both communicate with the expansion fluid port 71 (FIG. 2). When the expansion fluid is supplied from the expansion fluid port 71, the proximal end portion expansion lumen 23L supplies the expansion fluid into the proximal end portion of the balloon 3, and the distal end portion expansion lumen 24L supplies the expansion fluid into the distal end portion of the balloon 3. As a result, the balloon 3 starts to expand from both end portions (the proximal end portion and the distal end portion) before the intermediate portion 32 at the center in the axial direction.

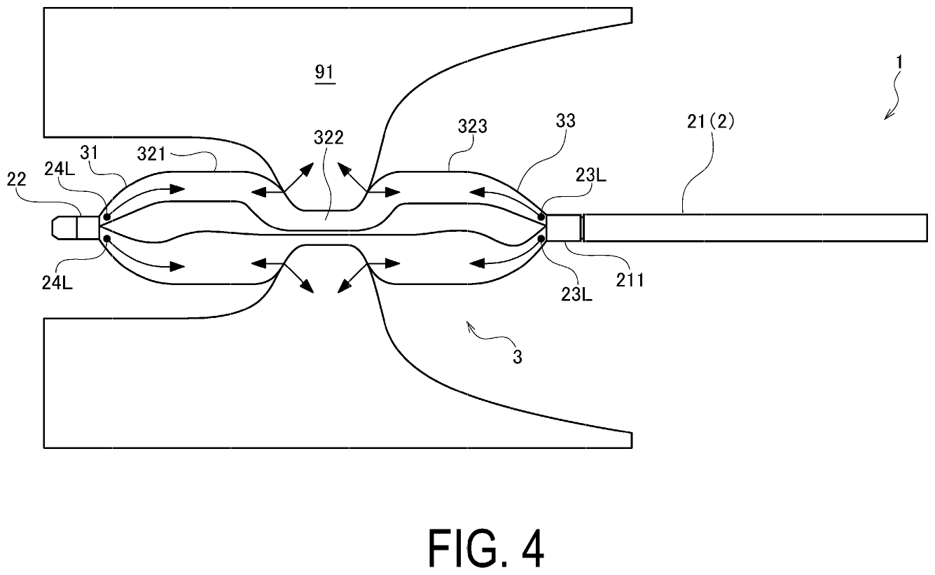

FIG. 4 schematically illustrates a state in which the balloon 3 of the balloon catheter 1 according to the present embodiment is positioned with respect to the papilla 91 as a dilatation target part. The balloon 3, having been guided by the guide wire 6 (not illustrated) and having reached the position of the papilla 91, starts expansion by the expansion fluid supplied from the expansion fluid port 71 (not illustrated). The expansion fluid is supplied into the balloon 3 simultaneously from the open end of the distal end portion expansion lumen 24L (distal end surface 21D) at the distal end side of the balloon 3 (in the distal end side tapered portion 31) and the open end of the proximal end portion expansion lumen 23L (proximal end surface 21P) at the proximal end side of the balloon 3 (in the proximal end side tapered portion 33). Therefore, the balloon 3 starts to expand from the distal end portion and the proximal end portion. Since the intermediate portion 32 is clamped by the papilla 91 in the radial direction, a narrow portion 322 is formed in the intermediate portion 32 immediately after the balloon 3 starts to expand.

The papilla 91 is supported from both sides in the axial direction by both end portions (shoulder portions) which have been expanded earlier. Therefore, during expansion of the balloon 3, the relative positional relationship between the narrow portion 322 (waist portion) and the papilla 91 does not change. That is, the narrow portion 322 of the balloon 3 is positioned with respect to the papilla 91 as a dilatation target part. Further, when the expansion fluid is continuously supplied into the balloon 3 having expanded into an hourglass shape from at least one of the distal end portion expansion lumen 24L and the proximal end portion expansion lumen 23L, the narrow portion 322 is expanded later than both end portions. As a result, the papilla 91 is dilated.

At this time, the narrow portion 322 contracts (the diameter increases and the difference in diameter from both ends decreases) in response to an increase in the amount and/or pressure of the expansion fluid in the internal space 21B of the balloon 3, and eventually substantially disappears as illustrated in FIG. 3 (that is, the intermediate portion 32 expands into a substantially straight tubular shape substantially devoid of the narrow portion 322. However, when the maximum expansion diameter of the papilla 91 is small, the narrow portion 322 may remain). Here, the “distal end portion” of the balloon 3 which is preferentially expanded by the distal end portion expansion lumen 24L includes not only the distal end side tapered portion 31 but also a distal end side straight tube portion 321 of the intermediate portion 32. Similarly, the “proximal end portion” of the balloon 3 which is preferentially expanded by the proximal end portion expansion lumen 23L includes not only the proximal end side tapered portion 33 but also a proximal end side straight tube portion 323 of the intermediate portion 32.

In the state of FIG. 4, the papilla 91 is sandwiched and supported from both sides by the distal end side straight tube portion 321 and the proximal end side straight tube portion 323. At this time, as schematically illustrated by arrows at the contact portion between the balloon 3 and the papilla 91, the distal end side straight tube portion 321 receives a reaction force (reaction) in a direction from the surface at the distal end side of the papilla 91 toward the distal end side, and the proximal end side straight tube portion 323 receives a reaction force (reaction) in a direction from the surface at the proximal end side of the papilla 91 toward the proximal end side. In this manner, when the balloon 3 is positioned with respect to the papilla 91, the balloon 3 is subjected to reaction forces in opposite directions from the papilla 91. Since the reaction forces in these directions weaken each other, the reaction force in the axial direction subjected to the balloon 3 from the papilla 91 is weak or substantially zero. Therefore, when the balloon 3 is positioned with respect to the papilla 91, the positional shift of the balloon 3 in the axial direction due to the reaction from the papilla 91 is effectively prevented. On the other hand, in a typically known balloon catheter, since the expansion fluid is supplied only to the proximal end portion of the balloon, there is the problem that the balloon expanded from the proximal end portion is subjected to a strong reaction in a direction from the dilatation target part such as the papilla toward the proximal end side and is moved backward in a direction opposite to the insertion direction.

In addition, when wishing to match the formation position of the narrow portion 322 with the central portion in the axial direction of the balloon 3 as illustrated in FIG. 4, for example, even if the expansion fluid is simultaneously supplied from the single expansion fluid port 71 to the distal end portion expansion lumen 24L and the proximal end portion expansion lumen 23L, there is a possibility that the expansion fluid is not supplied into the balloon 3 from the expansion lumens 24L and 23L at the same time. Specifically, as illustrated in FIG. 3, since the distal end portion expansion lumen 24L extends more than the proximal end portion expansion lumen 23L by the axial distance between the proximal end surface 21P and the distal end surface 21D in the balloon 3, there is a possibility that the expansion fluid is supplied into the balloon 3 from the distal end portion expansion lumen 24L later than the when the expansion fluid is supplied into the balloon 3 from the proximal end portion expansion lumen 23L.

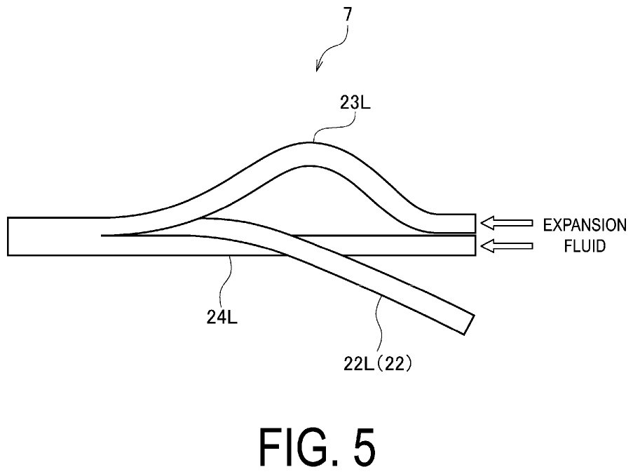

As schematically illustrated in FIG. 5, in order to eliminate the discrepancy in length between the distal end portion expansion lumen 24L and the proximal end portion expansion lumen 23L, the proximal end portion expansion lumen 23L may be formed longer than the distal end portion expansion lumen 24L in the port portion 7 at the proximal end side of the shaft 2 by an amount corresponding to the difference in length in the axial direction (axial distance) from the distal end portion expansion lumen 24L in the balloon 3. That is, the proximal end portion expansion lumen 23L is formed with a surplus in the port portion 7 that serves as a fluid supply portion for supplying the expansion fluid to the proximal end portions of the distal end portion expansion lumen 24L and the proximal end portion expansion lumen 23L.

On the other hand, in the case where the discrepancy in length between the distal end portion expansion lumen 24L and the proximal end portion expansion lumen 23L in FIG. 3 does not cause a practical problem (for example, in the case where the discrepancy can be ignored with respect to the entire length of the shaft 2), there is little need to form the proximal end side of the outer shaft 21 into an expensive multi-lumen structure including the distal end portion expansion lumen 24L and the proximal end portion expansion lumen 23L. Therefore, the distal end portion expansion lumen 24L and the proximal end portion expansion lumen 23L may be an inexpensive single lumen on the proximal end side of the proximal end portion of the balloon 3 in the outer shaft 21 (for example, between the proximal end of the outer shaft 21 and the balloon attachment portion 211). However, it is preferable to provide the central lumen 21L for the inner shaft 22 inside the outer shaft 21 separately from the single lumen.

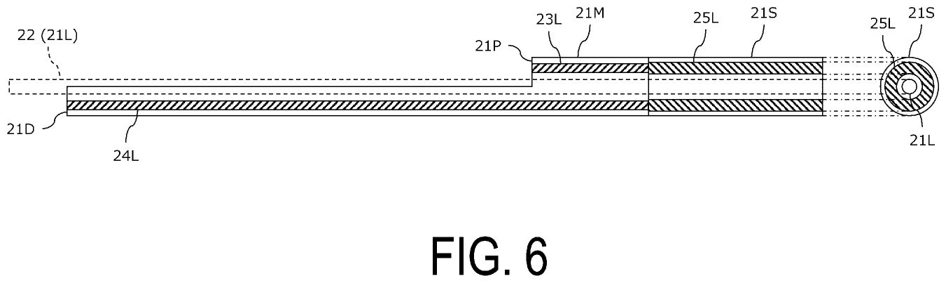

Specifically, as schematically illustrated in FIG. 6, a portion of the outer shaft 21 to which the balloon 3 is attached at the distal end side (left side in FIG. 6) is a multi-lumen shaft 21M in which the above-described two types of expansion lumens 23L and 24L are individually formed. On the other hand, a portion of the outer shaft 21 at the proximal end side (right side in FIG. 6) to which the balloon 3 is not attached is a single lumen shaft 21S in which one type of expansion lumen 25L is formed. As schematically illustrated in the right side of FIG. 6, in the cross-section of the single lumen shaft 21S, the expansion lumen 25L is a substantially annular single lumen surrounding the central lumen 21L at the center. The distal end of the expansion lumen 25L communicates with the proximal ends of the two types of expansion lumens 23L and 24L of the multi-lumen shaft 21M. In other words, at the contact surface between the single lumen shaft 21S and the multi-lumen shaft 21M, the single expansion lumen 25L branches into two types of expansion lumens 23L and 24L. The expansion lumen 25L communicates with the expansion fluid port 71 (FIG. 2) at the proximal end portion and supplies the expansion fluid to the two types of expansion lumens 23L and 24L at the distal end portion. According to this modification, the same advantageous effects as those of the balloon catheter 1 can be expected.

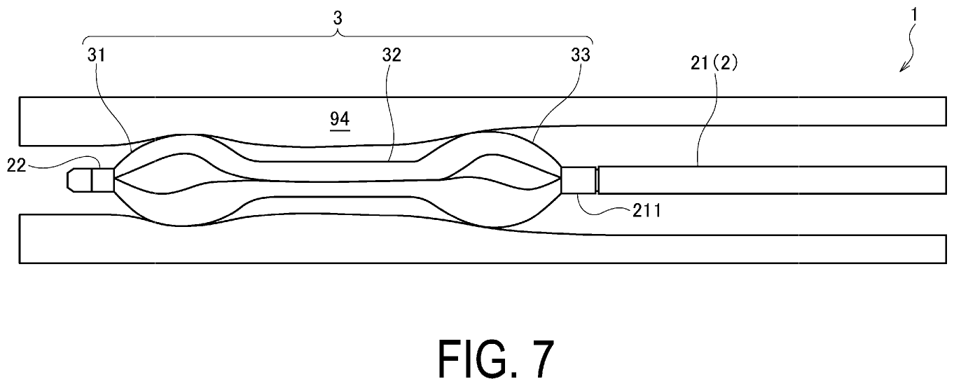

FIG. 7 schematically illustrates a state in which the balloon 3 of the balloon catheter 1 according to the present embodiment is positioned with respect to a constricted part 94 as a dilatation target part. The balloon 3, having been guided by the guide wire 6 (not illustrated) and having reached the position of the constricted part 94, starts expansion by the expansion fluid supplied from the expansion fluid port 71 (not illustrated). Similarly as in FIG. 4, the balloon 3 starts to expand from the distal end side tapered portion 31 and the proximal end side tapered portion 33, and expands into the hourglass shape in which the intermediate portion 32 is narrow.

The distal end side tapered portion 31 and the proximal end side tapered portion 33 which have expanded earlier than the intermediate portion 32 are positioned in contact with a vessel wall at the front and back of the constricted part 94. Further, when the expansion fluid is continuously supplied into the balloon 3 having expanded into the hourglass shape from at least one of the distal end portion expansion lumen 24L and the proximal end portion expansion lumen 23L, the intermediate portion 32 is expanded later than both end portions, and thus the constricted part 94 as a dilatation target part is dilated.

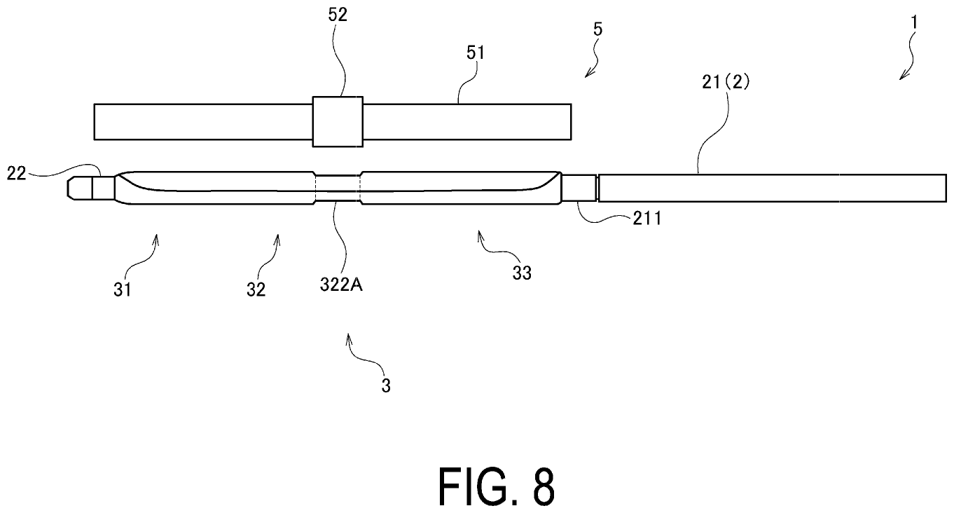

FIG. 8 schematically illustrates an example of a balloon molding tool 5 for effectively forming the narrow portion 322 as illustrated in FIG. 4. The balloon molding tool 5 is a heat-shrinkable tube for performing a molding process or a pressurizing process accompanied by heating on the balloon 3 folded and wound around the outer periphery of the shaft 2. The balloon molding tool 5 which is heated and contracted in a state where the balloon 3 wound around the shaft 2 is inserted molds the balloon 3 into a folded and completely contracted shape.

The balloon molding tool 5 includes a molding enhancing portion 52 at a central portion in an axial direction of a tubular tube main body 51. The molding enhancing portion 52 is disposed in a central portion (the intermediate portion 32) of the balloon 3 where the narrow portion 322 is to be formed. When the balloon molding tool 5 is heated in this state, the central portion (the intermediate portion 32) of the balloon 3 where the molding enhancing portion 52 is disposed is molded more strongly than the distal end portion and the proximal end portion. For example, the central portion of the balloon 3 is molded at a higher pressure than the distal end portion and the proximal end portion by the molding enhancing portion 52 which contracts more than the tube main body 51. Alternatively, the central portion of the balloon 3 is heated more than the distal end portion and the proximal end portion by the molding enhancing portion 52 having higher thermal conductivity than the tube main body 51. As a result, an expansion resistance structure 322A is formed in the central portion of the balloon 3 which is strongly molded by the molding enhancing portion 52.

The expansion resistance structure 322A formed in the central portion of the balloon 3 gives a larger resistance to the expansion pressure by the expansion fluid than the distal end portion and the proximal end portion. Therefore, as illustrated in FIG. 4, the balloon 3 starts to expand from the distal end portion and the proximal end portion where the expansion resistance is small, and the narrow portion 322 is effectively formed in the expansion resistance structure 322A where the expansion resistance is large. FIG. 8 schematically illustrates the expansion resistance structure 322A strongly molded by the molding enhancing portion 52 as a portion having a smaller diameter than other portions of the balloon 3, but the result of the strong molding by the molding enhancing portion 52 does not necessarily appear as a difference in diameter. For example, the result of the strong molding by the molding enhancing portion 52 may appear as a difference in molecular structure such as orientation of molecules in the expansion resistance structure 322A, a difference in residual stress, a difference in shape, or a difference in any other property or state.

The expansion resistance structure 322A in the central portion of the balloon 3 may be configured with surface properties of the balloon 3 different from those of the distal end portion and the proximal end portion. For example, the expansion resistance structure 322A can be made by forming the outer peripheral surface and/or the inner peripheral surface of the central portion of the balloon 3 into a smooth surface or the like having higher tackiness or adhesiveness than the distal end portion and the proximal end portion. The expansion resistance structure 322A can be also made by forming irregularities for applying resistance due to meshing, friction, or the like on the outer peripheral surface and/or the inner peripheral surface of the central portion of the balloon 3. The expansion resistance structure 322A may be made by applying or adding an adhesive or the like that imparts expansion resistance to the outer peripheral surface and/or the inner peripheral surface of the central portion of the balloon 3 as long as the treatment by the balloon catheter 1 is not hindered.

A second embodiment of the present disclosure will be described. The balloon catheter according to the second embodiment has a portion having a similar structure to that of the balloon catheter according to the first embodiment. Portions having the same structure as those of the balloon catheter according to the first embodiment are denoted with the same reference signs as those of the first embodiment, and detailed description thereof is omitted.

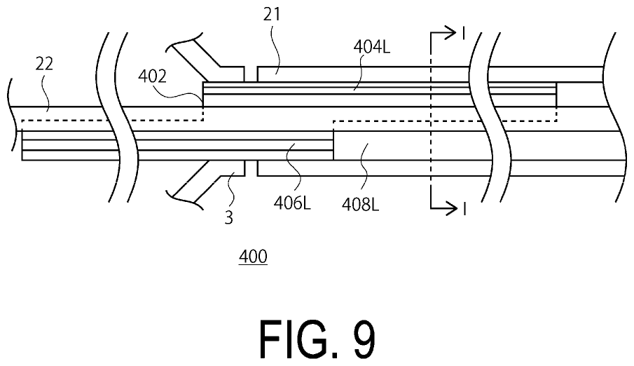

FIG. 9 is a cross-sectional view of the balloon catheter according to the second embodiment. A balloon catheter 400 according to the second embodiment includes a flow velocity adjustment portion 402 near the distal end of the outer shaft 21. The flow velocity adjustment portion 402 adjusts the flow velocity of an expansion fluid flowing into the outer shaft 21 from expansion fluid port 71.

At least one proximal end portion expansion lumen 404L is formed in the flow velocity adjustment portion 402. At least one distal end portion expansion lumen 406L in which the positions along the axial direction of a start point (i.e., inlet of expansion fluid) and an end point (i.e., outlet of expansion fluid) are different from the positions of a start point and an end point of the proximal end portion expansion lumen 404L is further formed in the flow velocity adjustment portion 402. Note that in the present embodiment, the inlet of the expansion fluid refers to an opening of the flow velocity adjustment portion 402 at an upstream side of the flow of the expansion fluid when expanding the balloon 3, and the outlet of the expansion fluid refers to an opening of the flow velocity adjustment portion 402 on a downstream side of the flow of the expansion fluid when expanding the balloon 3.

The inlet of the proximal end portion expansion lumen 404L is at the proximal end side relative to the inlet of the distal end portion expansion lumen 406L. The outlet of the proximal end portion expansion lumen 404L is at the proximal end side relative to the outlet of the distal end portion expansion lumen 406L. That is, when expanding the balloon 3, the expansion fluid enters the proximal end portion expansion lumen 404L at an earlier time.

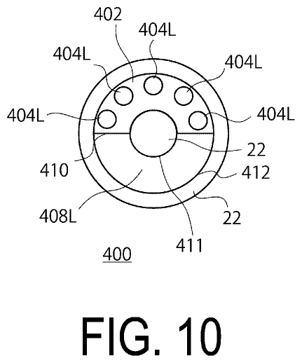

FIG. 10 is a cross-sectional view along line I-I in FIG. 9. Specifically, FIG. 10 illustrates a cross section of an area between the distal end portion expansion lumen 406L and the proximal end portion expansion lumen 404L. As illustrated in FIG. 10, this cross section has a plurality of the proximal end portion expansion lumens 404L (each lumen corresponds to a “first region”) and another lumen (hereinafter, referred to as “semicircular lumen 408L”, which corresponds to a “second region”). The semicircular lumen 408L is defined by a substantially flat wall surface 410 of the flow velocity adjustment portion 402, an outer peripheral surface 411 of the inner shaft 22, and a curved surface 412 of the outer shaft 21. Note that in the illustrated example, the semicircular lumen 408L does not have a semicircular shape, but has a shape (also referred to as half ring shape) where a semicircular shape having a small diameter has been removed from a semicircular shape having a large diameter. However, an important point in the second embodiment is a relative relationship between the velocity of the fluid flowing through the semicircular lumen 408L and the velocity of the fluid flowing through the proximal end portion expansion lumen 404L. From the viewpoint of the relative relationship of the velocity of the fluid, even if the shape of the semicircular lumen 408 is not a true semicircular shape, substantially no problem occurs, and thus the term “semicircle” is used in the present embodiment.

Although detailed principles will be described later, the structure (i.e., the flow velocity adjustment portion 402 and the outer shaft 21) that defines the proximal end portion expansion lumen 404L and the semicircular lumen 408L forms a flow velocity adjustment mechanism that adjusts the flow velocity of the expansion fluid as a whole.

Detailed description will be given focusing on a ratio of an open edge length and a cross-sectional area of each of one proximal end portion expansion lumen 404L and the semicircular lumen 408L. Dimensions of the one proximal end portion expansion lumen 404L and the semicircular lumen 408L are determined such that the frictional resistance in the proximal end portion expansion lumen 404L becomes greater than the frictional resistance in the semicircular lumen 408L by adjusting the ratio of the open edge length to the cross-sectional area. As a result, the velocity of the expansion fluid flowing in the proximal end portion expansion lumen 404L becomes slower than the velocity of the expansion fluid flowing in the semicircular lumen 408L. As a result, if the flow rate of the expansion fluid is constant, the relative relationship between the timing at which the expansion fluid enters the inlet of the distal end portion expansion lumen 406L and the timing at which the expansion fluid enters the inlet of the proximal end portion expansion lumen 404L can be adjusted. If the length of the proximal end portion expansion lumen 404L is increased, the timing at which the expansion fluid is discharged from the proximal end portion expansion lumen 404L is delayed in proportion to the length of the proximal end portion expansion lumen 404L. On the other hand, if the length of the distal end portion expansion lumen 406L is reduced, the timing at which the expansion fluid is discharged from the distal end portion expansion lumen 406L is advanced in inverse proportion to the length of the distal end portion expansion lumen 406L. That is, by adjusting the velocity of the expansion fluid and the flow distance (the length of the distal end portion expansion lumen 406L, the length of the semicircular lumen 408L, and the length of the proximal end portion expansion lumen 404L) of the expansion fluid, it is possible to adjust the timing at which the expansion fluid is discharged from the distal end portion expansion lumen 406L and the timing at which the expansion fluid is discharged from the proximal end portion expansion lumen 404L. In a preferred example, the length of the proximal end portion expansion lumen 404L is shorter than the length of the distal end portion expansion lumen 406L. As a result, it is possible to delay the timing at which the expansion fluid flowing through the proximal end portion expansion lumen 406L is discharged while advancing the timing at which the expansion fluid flowing through the distal end portion expansion lumen 404L is discharged.

When the timing at which the expansion fluid is discharged from the distal end portion expansion lumen 406L is made to be simultaneous with or slightly earlier than the timing at which the expansion fluid is discharged from the proximal end portion expansion lumen 404L, the distal end side and the proximal end side of the balloon 3 can be almost simultaneously expanded. As a result, for example, when the papilla 91 is expanded, the balloon 3 can be suppressed from moving to the proximal end side or the distal end side.

Note that in the above example, the description has been given under the assumption that the diameter of the distal end portion expansion lumen 406L and the diameter of the proximal end portion expansion lumen 404L are the same. That is, it is assumed that the velocity of the expansion fluid flowing through the distal end portion expansion lumen 406L and the velocity of the expansion fluid flowing through the proximal end portion expansion lumen 404L become the same. However, the timing at which the expansion fluid is discharged from the lumen may be controlled by adjusting various parameters such as the diameter, the length, the cross-sectional area, the open edge length, and the shape of the proximal end portion expansion lumen 404L and the distal end portion expansion lumen 406L. The semicircular lumen may be omitted depending on the diameter and the length of the distal end portion expansion lumen 406L and the proximal end portion expansion lumen 404L.

The present disclosure has been described above based on the embodiments. It is obvious to those skilled in the art that various modifications can be made to the combination of the components and processes in the exemplary embodiments and that such modifications are included in the scope of the present disclosure.

Note that the configuration, action, and function of each device and each method described in the embodiments can be implemented by hardware resources, software resources or in cooperation of hardware resources and software resources. For example, processors, ROMs, RAMs, and various integrated circuits can be used as the hardware resources. For example, programs such as operating systems and applications can be used as the software resources.

While preferred embodiments of the present disclosure have been described above, it is to be understood that variations and modifications will be apparent to those skilled in the art without departing from the scope and spirit of the disclosure. The scope of the present disclosure, therefore, is to be determined solely by the following claims.

1. A balloon catheter comprising:
a shaft that extends in an axial direction from a proximal end side to a distal end side; and
a balloon that is attached to the distal end side of the shaft and is expandable by a fluid supplied from the proximal end side of the shaft, wherein
the shaft internally includes a distal end portion expansion lumen for supplying the fluid to a distal end portion in the balloon, and a proximal end portion expansion lumen for supplying the fluid to a proximal end portion in the balloon.

2. The balloon catheter according to claim 1, wherein an intermediate portion between the distal end portion and the proximal end portion is provided with an expansion resistance structure that gives a larger resistance to an expansion pressure caused by the fluid than the distal end portion and the proximal end portion.

3. The balloon catheter according to claim 2, wherein, in the expansion resistance structure, a diameter of the intermediate portion is smaller than a diameter of the distal end portion and a diameter of the proximal end portion in a contracted shape of the balloon before expansion.

4. The balloon catheter according to claim 2, wherein the expansion resistance structure in the intermediate portion is formed with a surface property of the balloon being different from a surface property of the distal end portion and a surface property of the proximal end portion.

5. The balloon catheter according to claim 1, wherein
a portion of the shaft in which the distal end portion expansion lumen is provided terminates at the distal end portion in the balloon, and
a portion of the shaft in which the proximal end portion expansion lumen is provided terminates at the proximal end portion in the balloon.

6. The balloon catheter according to claim 1, wherein the proximal end portion expansion lumen is formed to be longer than the distal end portion expansion lumen in a fluid supply portion that supplies the fluid to the distal end portion expansion lumen and a proximal end portion of the proximal end portion expansion lumen.

7. The balloon catheter according to claim 1, wherein
a single expansion lumen through which the fluid supplied from a proximal end side of the shaft passes is provided at a proximal end side of the proximal end portion of the balloon in the shaft, and
the single expansion lumen communicates with the distal end portion expansion lumen and the proximal end portion expansion lumen at a distal end side of the proximal end portion of the balloon in the shaft.

8. A balloon expansion method comprising simultaneously supplying a fluid from a distal end portion and a proximal end portion into a balloon to expand the balloon into an hourglass shape having a narrow portion in an intermediate portion between the distal end portion and the proximal end portion.

9. The balloon expansion method according to claim 8, comprising, after expanding the balloon into the hourglass shape, further supplying the fluid into the balloon to reduce a difference in diameter between the narrow portion, the distal end portion, and the proximal end portion.

10. A balloon catheter comprising:
a shaft that extends in an axial direction from a proximal end side to a distal end side; and
a balloon that is attached to the distal end side of the shaft and is expandable by a fluid supplied from the proximal end side of the shaft, wherein
the shaft includes, in a cross section orthogonal to the axial direction, a first region in which a fluid flows in the axial direction at a first velocity and a second region in which a fluid flows in the axial direction at a second velocity different from the first velocity.
